(19) United States
(12) Patent Application Publication
(10) Pub. No.: US 2013/0144215 A1
(43) Pub. Date: Jun. 6, 2013

(54) Medicine Dispensing Device

(71) Applicants: Victor Esteve, Sao Paulo (BR); Eric Zembrod, Boituva (BR)

(72) Inventors: Victor Esteve, Sao Paulo (BR); Eric Zembrod, Boituva (BR)

(21) Appl. No.: 13/639,282

(22) PCT Filed: Mar. 4, 2011
(86) PCT No.: PCT/BR2011/000066

§ 371 Date: Oct. 4, 2012

(30) Foreign Application Priority Data
Mar. 8, 2010 (BR) PI1000731-8

(51) Int. Cl. A61M 11/00

(52) U.S. Cl. CPC A61M 11/00
USPC 604/151

(57) ABSTRACT

Medicine dispensing device, comprising a body (8) defined by two parts, a grip (9) and a head (10), coupled together by means (11) existing on their edges which face each other, these two parts (9-10) ordinarily being elliptical, the first vertically elongated, whereas the second is substantially short, and also has its upper side closed (12) with a fitting perimeter (13) for a lid (14) which also covers a raised and hollowed out central part (15) which, on the outside shows the dispensing actuator nozzle (7) and, on the inside, presents suitable means (16) to house the actuator (5), principally the flange (6), beneath which said flask (2) has its lower end fitted inside an elevator cup (17) which, in turn, is housed inside the grip (9), where sliders provide inner housing means (18) for the medicine packaging (1) and its elevator cup (17).

### FIELD OF THE INVENTION

More particularly the present invention refers to a drive mechanism in the form of outer-packaging, designed to internally receive a primary packaging traditionally used to contain a medicine normally in liquid state or having easy fluidity. This medicine is generally of the kind to be applied in spray form or drops, such as occurs in nasal treatment. In this condition, the aforementioned packaging also has an upper part in the form of an actuator internally valved to release metered doses in form of spray. This actuator is integrated with an internal valve system and metered dose pump which is driven by pressing said actuator which, accordingly, has a flange for this purpose. Hence, the present invention provides a body which, besides being an anatomical grip, includes in this same grip a trigger precisely to press the mechanism of the actuator and, consequently, drive said pump to release the output of the medicine in the form of spray.

A flask or medicine packaging is understood to be any recipient having a dispensing device with an actuator inside with a dosing pump and valve to dispense the medicine in the form of jets, whether or not in spray form.

### STATE OF THE ART

Currently there are different devices used for the same purpose, each with its constructive particularities, such as those taught in documents: BRPI9306950, BRPI0416128, BRPI0507383, DE19610456, FR2812826, FR2859464, FR2882349, FR2889691, FR2889692, JP10179739, U.S. Pat. No. 3,272,391, U.S. Pat. No. 4,771,769, U.S. Pat. No. 5,487,489, US2002170928, WO0249698, WO03061843A1, WO2005044354 and WO2005075103, WO2009068877.

There is no doubt that the above devices and others present suitable means to dispense the medicine, whether or not it is in the form of spray, however, in most cases, said mechanisms comprise a considerable number of articulated components and levers that, despite working satisfactorily, add details to complicate the assembly and the working of the assembly, consequently, considerably increasing its final cost as well.

Moreover, some of these devices, such as: DE19610456, FR2812826, DE19610456, WO2005075103 and WO2009068877, present as predominating characteristic a drive by levers that acts or depends directly on contact with a component of the dispensing pump as in its base, or adjacent parts as in the actuator and, also, in some cases, the mechanism requires the medicine flask itself to have some detail to be coupled to the drive mechanism. Obviously all this ultimately interferes in the industrial viability of the assembly, to define a complex assembly not only to produce, but also to assemble, consequently, the end cost also proportionally increases in relation to said advantages.

Another drawback of conventional mechanisms is, without a doubt, the limitations relating to the flask holding the medicine to be applied, because, it is normally specific for the corresponding drive device and does not allow, for example, the bottom of the flask to have different arrangements or different profile geometries.

Another factor also not found in the devices known in the art is the fact that they do not have resources or flexibility to adjust the drive strength and speed, which are significant for determining the spray pattern.

Therefore, the known devices present a considerable number of components, which requires longer assembly time. The same is true for the production of the components and, ultimately, besides characterizing a substantially complex mechanism, also present manufacturing costs which reflects the industrial complexity of its assembly.

### OBJECTIVES OF THE INVENTION

The device to dispense medicines was idealized with just two components responsible for driving the medicine flask pump. The first component is a trigger, combined with a second component which is a base in the form of a cup, which is a support cradle for fastening the bottom or lower end of the medicine flask. Said trigger has an ordinarily semicircular part which, from the outside, configures an anatomical finger support area for driving a press button, whereas from the inside said trigger, besides involving the medicine flask, also presents an elongated lower part in the form of two slanted arms, one on each side of the flask, whose lower ends each have an ordinarily horizontal protraction, where one of its ends is articulately integrated with the corresponding arm, and the median portion of said protraction has a cavity on the lower side of its articulated support on the corresponding inner side of the body, where said protraction moves like a seesaw (lever) and, further, its upper face describes a “cam surface” which supports radial projections existing on the edges of said cup, such that the latter can be displaced upwards or downwards, that is, when pressing the trigger the lower ends of its arms act on the corresponding ends of the seesaw protractions and, with this, its “cam surface” makes said cup be displaced upwards and downwards according to a sufficient extend to drive the pump of the medicine flask and dispense its content in the form of spray.

Therefore, it is noted that the device in question is defined merely by two mobile components, the trigger and the cup, where the first is a single injected component which combines portions specially designed and strategically positioned such that each portion can have one function and, thus, only a single component is capable of carrying out a succession of movements which ultimately displace said medicine flask upwards and, at this point, its pump mechanism is driven to dispense the medicine contained inside in a spray form. The succession of movements is the main characteristic of the mechanism in question, since, the part that acts as trigger is anatomically positioned and, when pressed inwards, makes the angular arms to be equally displaced, making its ends to be displaced downwards and, at this moment the second movement occurs, being the actuation of the “cam system”, that is, the protraction in seesaw form has its corresponding end displaced downwards, causing a seesaw (lever) effect and, with this, a third movement occurs on the “cam surface”, pushing the cup to be displaced upwards, causing the drive of the pump mechanism of the medicine flask.

An advantageous aspect of the present invention is, without a doubt, the drive button, because with a single component, it is possible to execute various consecutive movements to drive the medicine flask pump/valve mechanism, which is not the case in those devices disclaimed in the prior documents, where said movements depend on a higher number of components.

Another advantageous aspect of this system relates to the arrangement of. its upper portion, conceived to partially cover the upper portion of the dosing pump actuator, reducing its length, making its ergonomics safer to be introduced into the nostrils, even for pediatric use.

Another advantage provided by the present dispensing device, although optional, is that it provides means to exchange the empty medicine flask for a refill unit, which is carried out easily and quickly by using fast internal engagement and disengagement means on the flange of the actuator on the medicine flask, and these means are driven by pressing the corresponding sides of the outer-packaging upper portion.

### DESCRIPTION OF THE DRAWINGS

For improving the understanding, it follows a detailed description of the present invention, with references to the appended drawings:

FIGS. 1 to 3 represent, respectively, two perspectives and a side view, highlighting the details of a primary packaging of medicines with flask recipient and dosing pump/valve;

FIGS. 4 to 6 show, respectively, two perspectives of the device in question fully assembled and a perspective with its lid exploded, showing the dispensing mouthpiece of the medicine;

FIGS. 7 to 10 illustrate exploded perspectives from different angles, showing details of each component of the assembly;

FIG. 11 is a perspective of the assembly, a detailed cross-sectional view and two parts in exploded perspective, showing the grip and the top portion, as well as details of the coupling there between;

FIG. 12 illustrates an exploded perspective of the assembly and three details, in which the first is an angle view showing inner details of the upper portion and the other two details are cross-sections of the same angular view of the upper portion, highlighting the inner part which houses and retains the primary packaging shown in FIGS. 1, 2 and 3;

FIG. 13 is an overhead view with indications of cross-sections A-A and B-B;

FIGS. 14 and 15 are views of the cross-sections indicated in the prior figure, showing the medicine primary packaging housed inside the outer-mechanism in question;

FIG. 16 reproduces an exploded perspective and two expanded details of the elevator cup, in which is fitted the lower portion of the medicine flask;

FIG. 17 is an exploded perspective and an expanded detail of the grip, showing its inner side, highlighting the housing details on the inner lower portion of the mechanism and medicine packaging;

FIGS. 18 and 19 are different angle perspectives, showing details of the inner portion of the closure component of the mechanism;

FIG. 20 represents a cross-sectional side view, an exploded perspective and expanded details, highlighting the articulated trigger support;

FIG. 21 shows an exploded perspective only of the body and the trigger, showing its coupling tooth which establishes the limit of its return after it has been pressed;

FIG. 22 illustrates, respectively, a side view with the body in transparency, an exploded perspective and an expanded side view only of the trigger, highlighting details of the elevator cup drive, particularly the “cam system”;

FIG. 23 shows a side view of the assembled set, with the body cut view, highlighting the system in the actuated position, when components are driven by the trigger;

FIG. 24 reproduces, respectively, an expanded view of the outer-system inner lower portion, an exploded perspective of the assembly, and an expanded overhead view of lateral grip on the upper portion of the body, highlighting the constructive details which allows uncoupling means for use of refill;

FIGS. 26 to 28 show, respectively, two exploded perspectives with expanded details and a cross-sectional side view, highlighting a constructive variation for the articulated trigger support; and

FIGS. 29 to 31 also show, respectively, two exploded perspectives with expanded details and a cross-sectional side view, highlighting another constructive variation for the articulated trigger support.

### DETAILED DESCRIPTION OF THE INVENTION

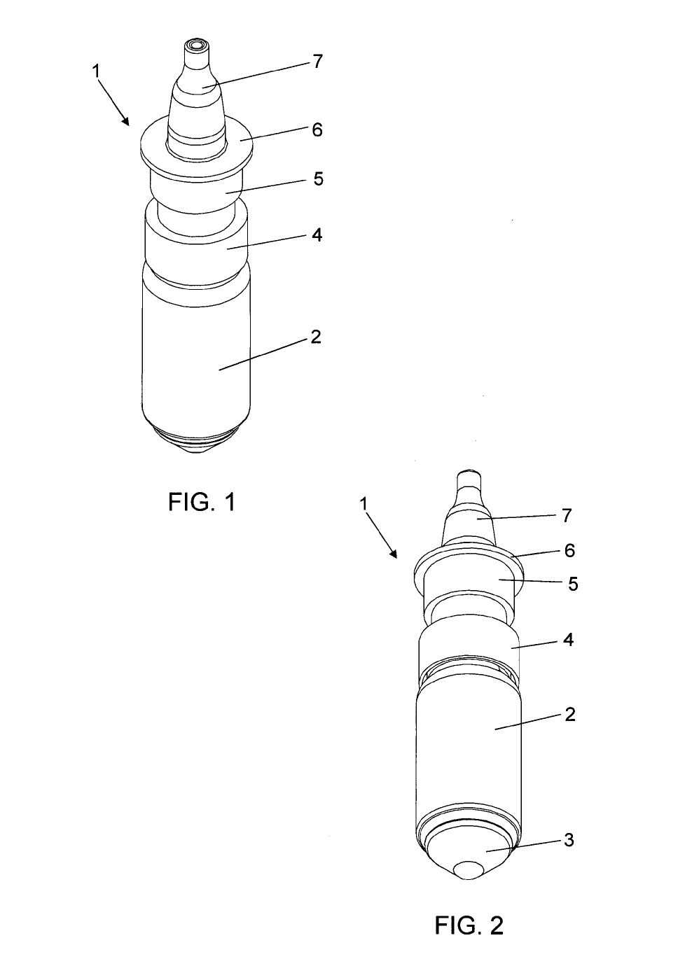

According to these illustrations and their details, the present MEDICINE DISPENSING DEVICE, as illustrated in FIGS. 1 to 3, is to be used with a traditional medicine primary packaging (1), comprising a flask (2), whose base or bottom (3) has a profile with any geometry, being it ogival, rounded or straight, whereas the upper part of said flask has a respective lid (4) which functions as an assembly base, having on its upper part an integrated actuator (5) with a median flange (6) and a dispensing portion(6), inside said actuator (5) being assembled a set what configures a dosing pump/valve (not illustrated) mechanism for dispensing the medicine contained in the packaging (1).

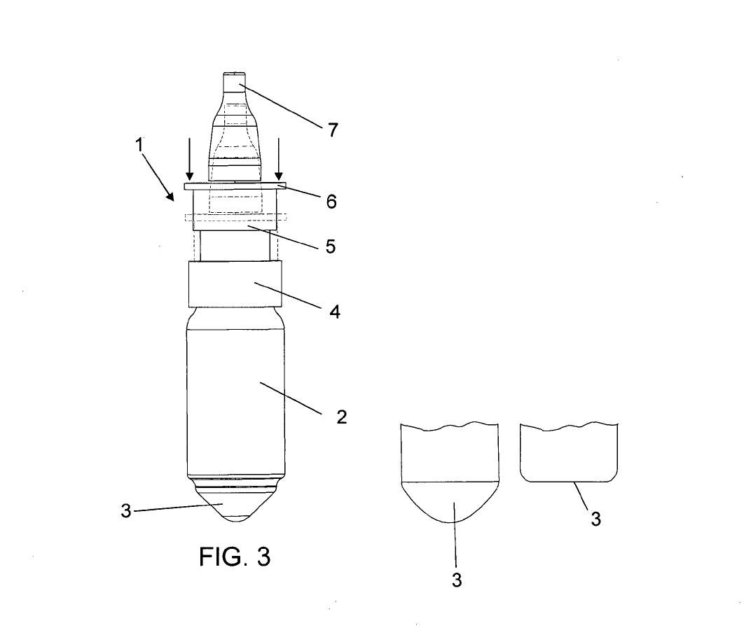

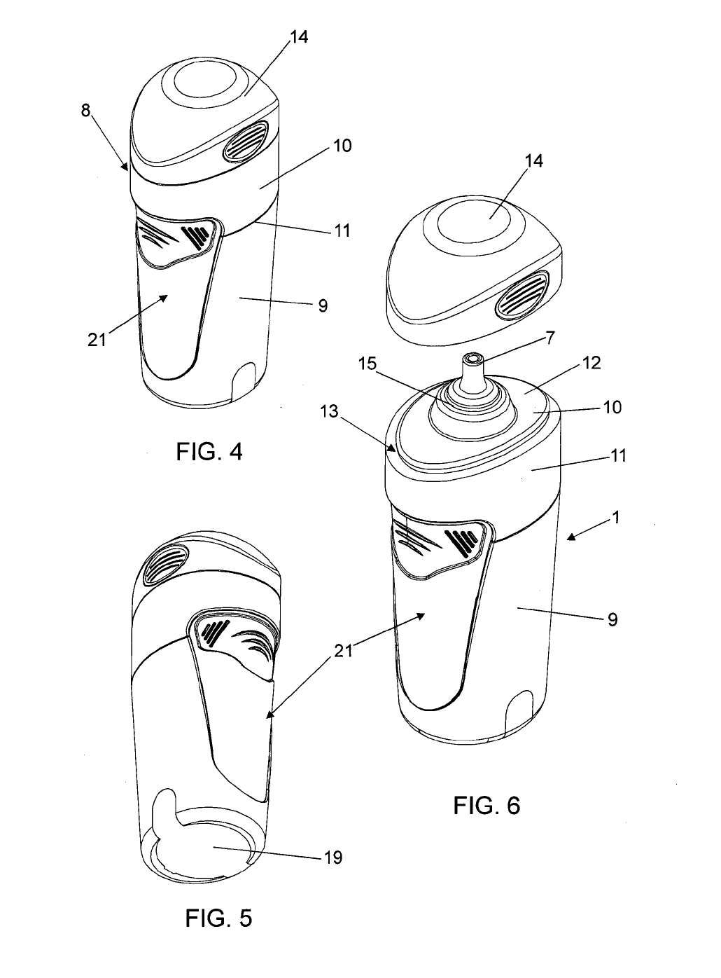

As indicated by the arrows in FIG. 3, the actuator (5) is downwardly displaceable and, this movement, makes the pump/valve start ejecting the medicine through the actuator nozzle (7) in the form of jets, either in spray or another suitable form for application, since, said assembly hereinafter identified as medicine packaging (1) may contain different types of medicines, such as those in liquid state or having easy fluidity, notably used for treating the eyes, ears, nose or throat.

Still referring to FIG. 3, the medicine packaging (1), by itself, is already a dispenser, since, pressing its flange (6) drives the pump/valve and, with this, an exact dose is ejected by the actuator nozzle (7) on the previously positioned site to be treated.

Although the medicine packaging (1) incorporates a drive which can be normally driven by its actuator (4), in many cases, a more anatomical form of drive is desirable, not only to improve the application of the medicine, but also to make the drive position more comfortable; inclusive the drive by using only the flask may present a certain complexity for users such as children, elderly or those who, for some reason, have poor motor skills. To solve these and other drawbacks and make the application safer, various drive devices have been created, such as all those listed previously, including that of the present invention, are designed to contain inside a primary medicine packaging (1) and, with the latter, combine means so that its flange or actuator can be driven with greater ease and comfort, making it recommended for adults and children.

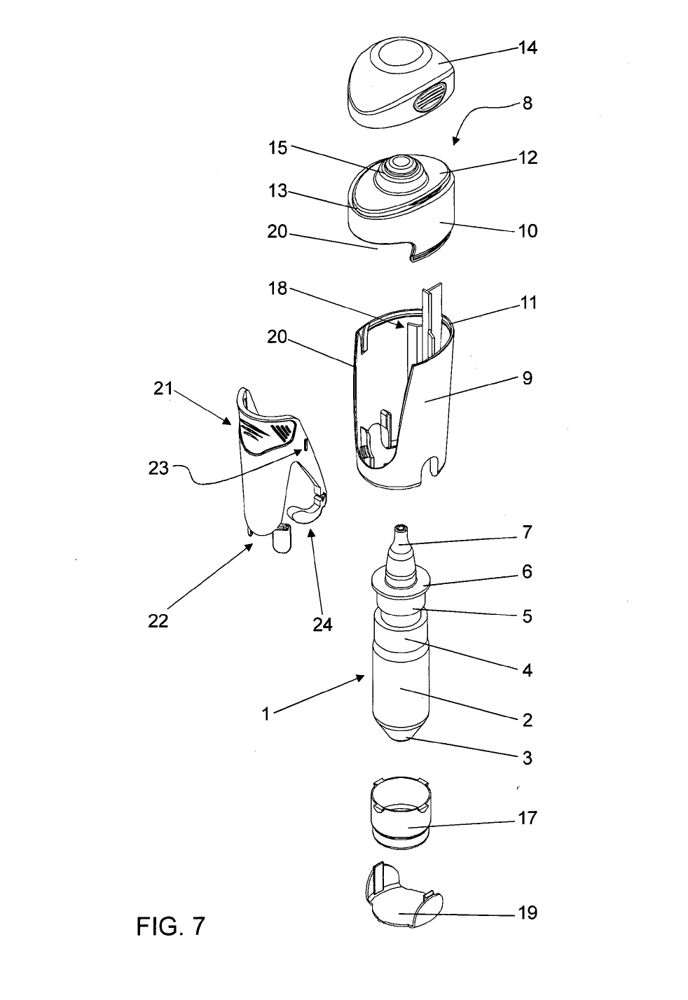

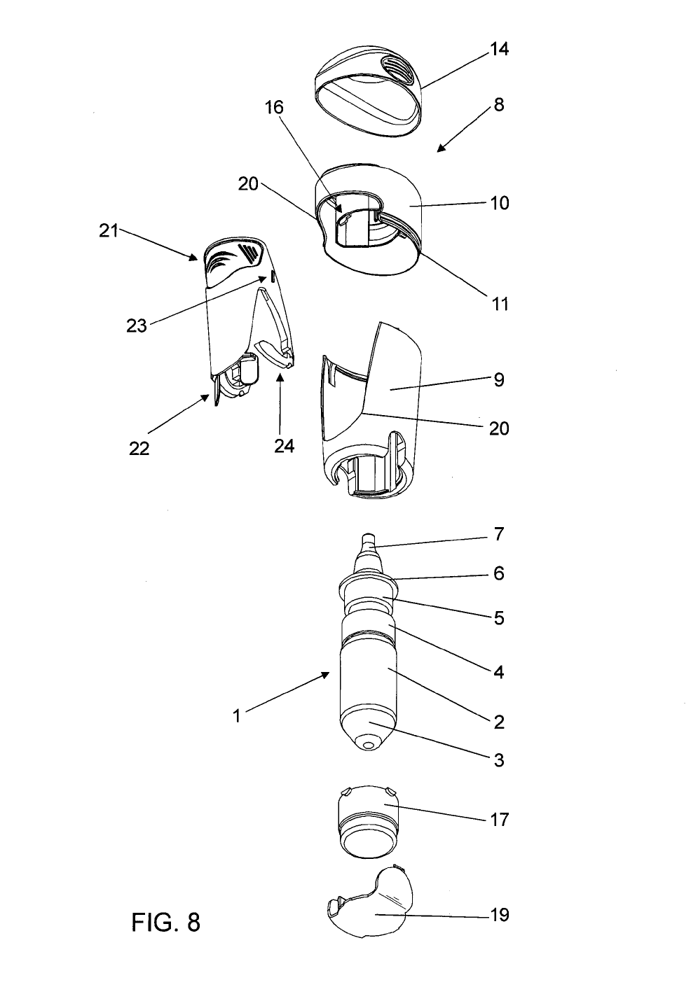

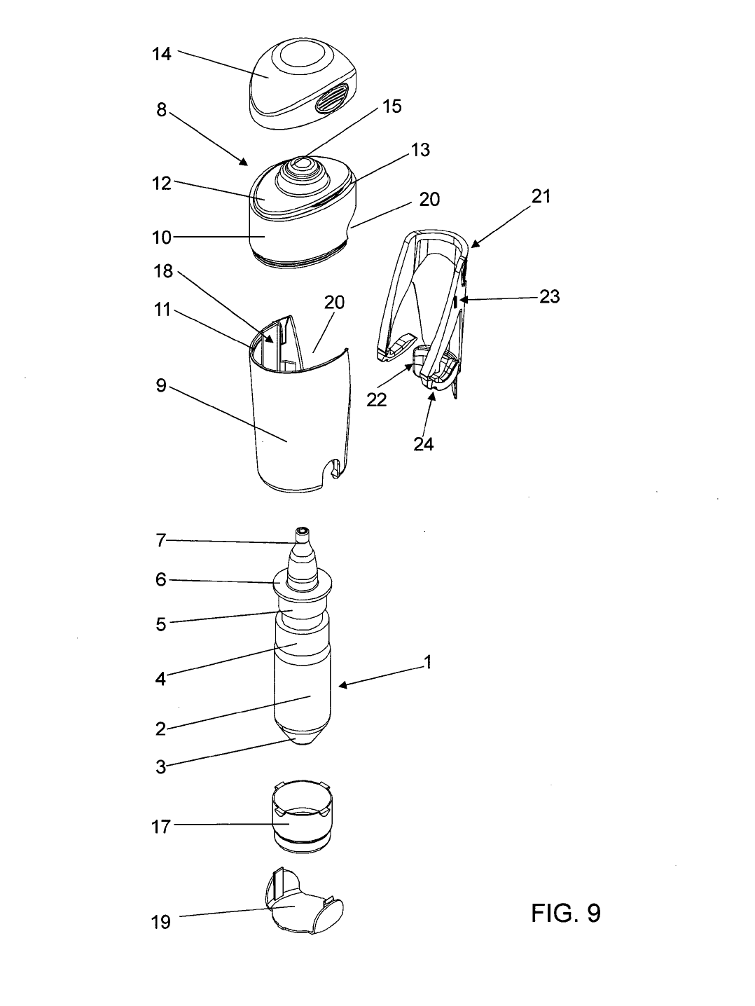

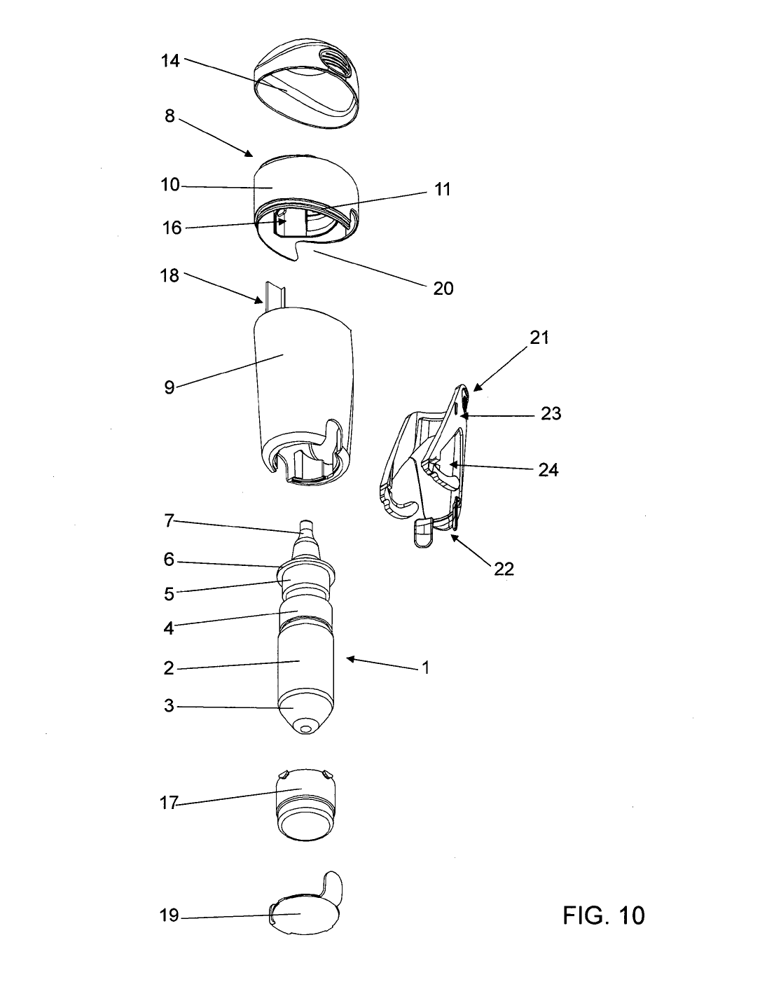

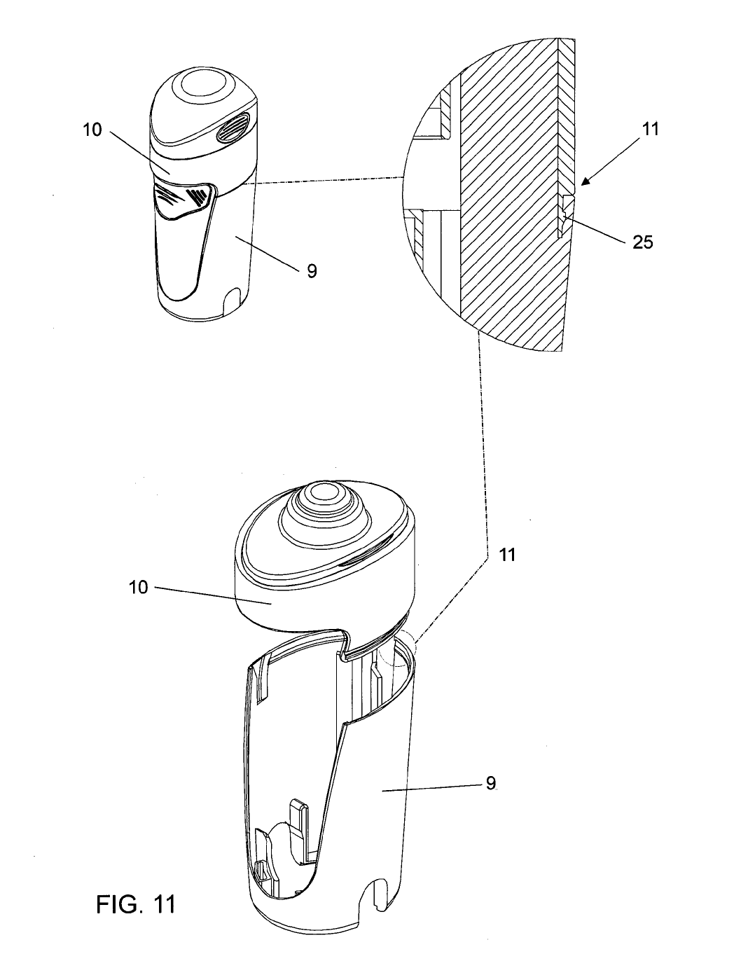

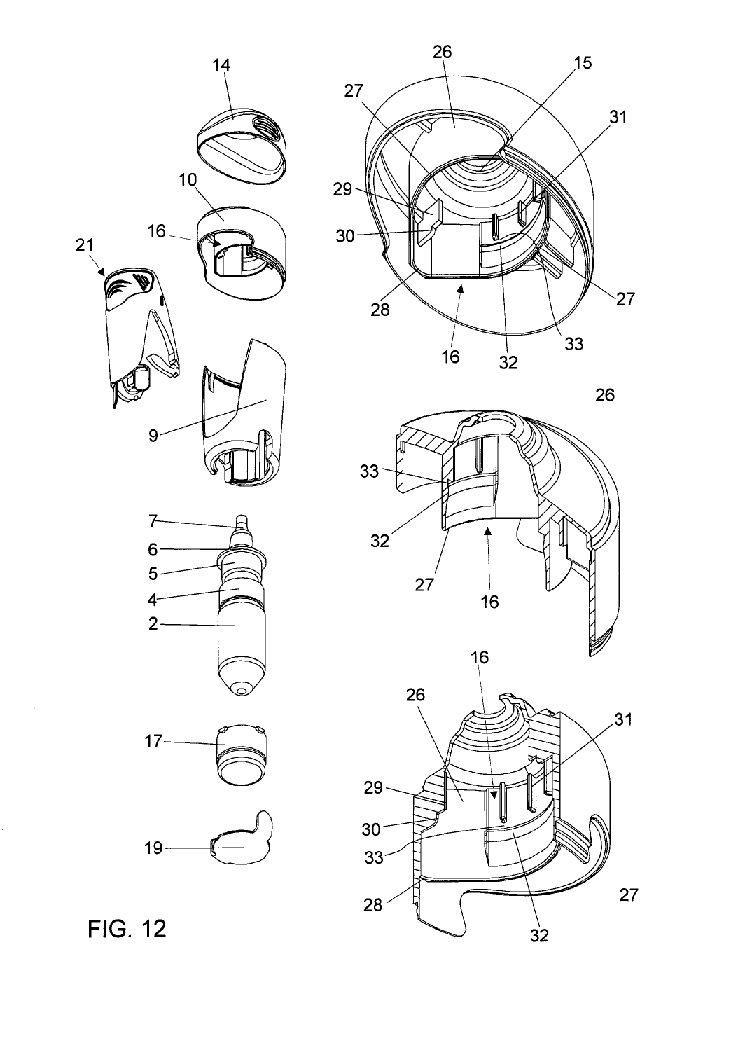

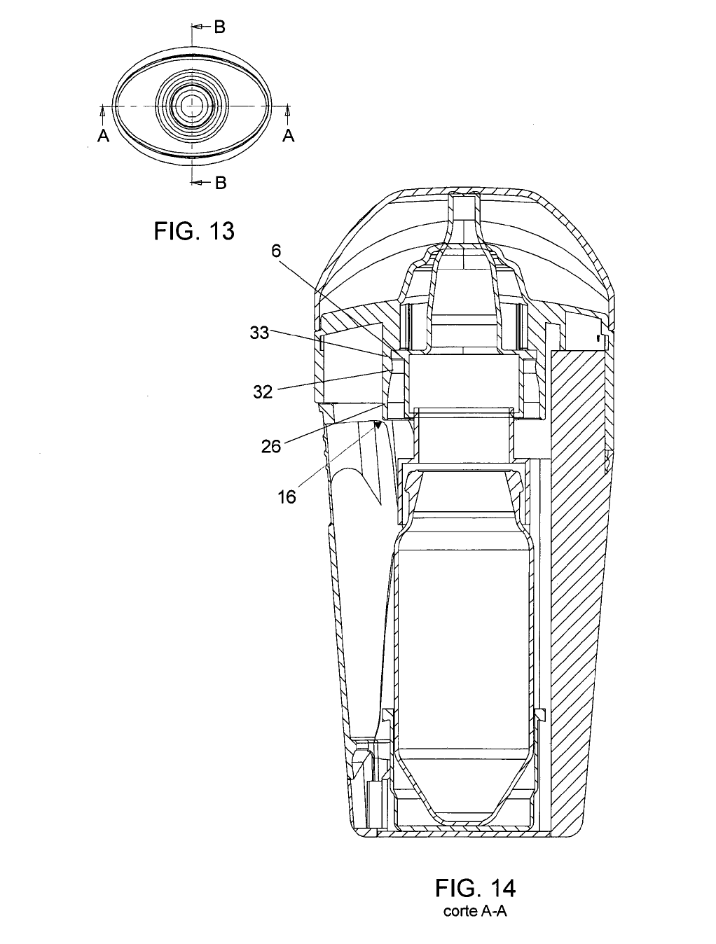

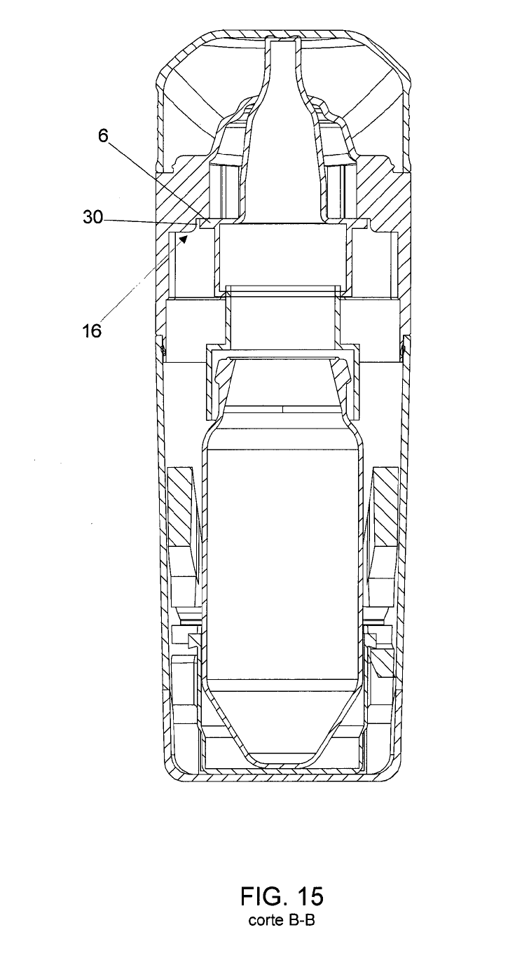

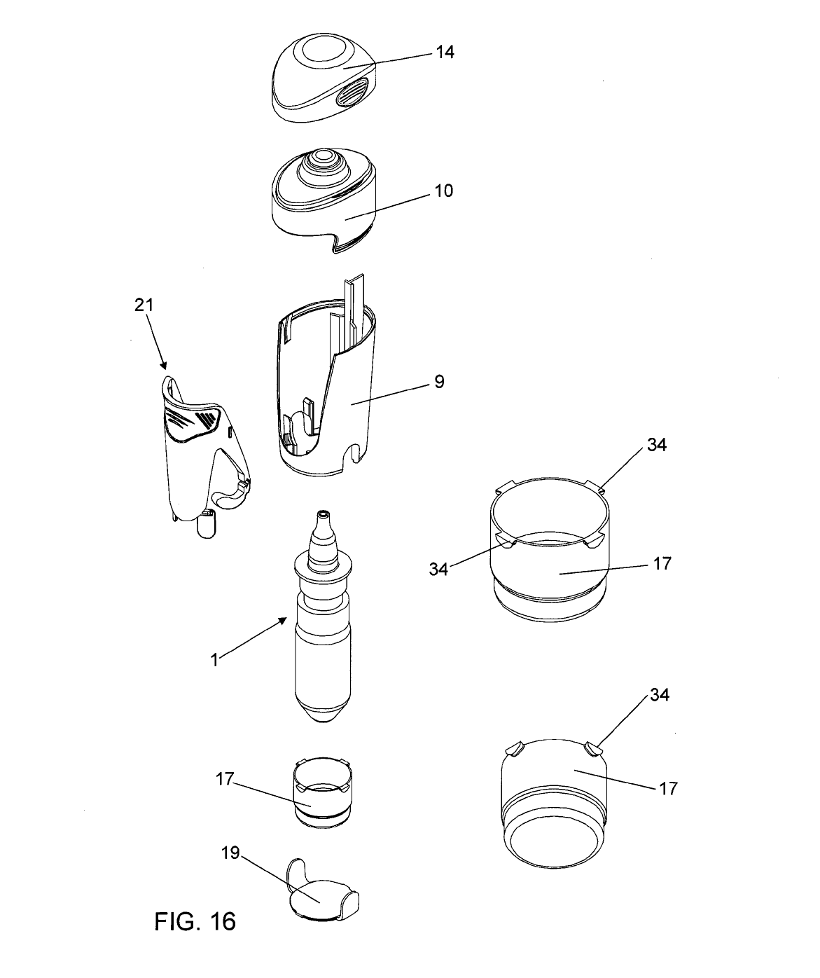

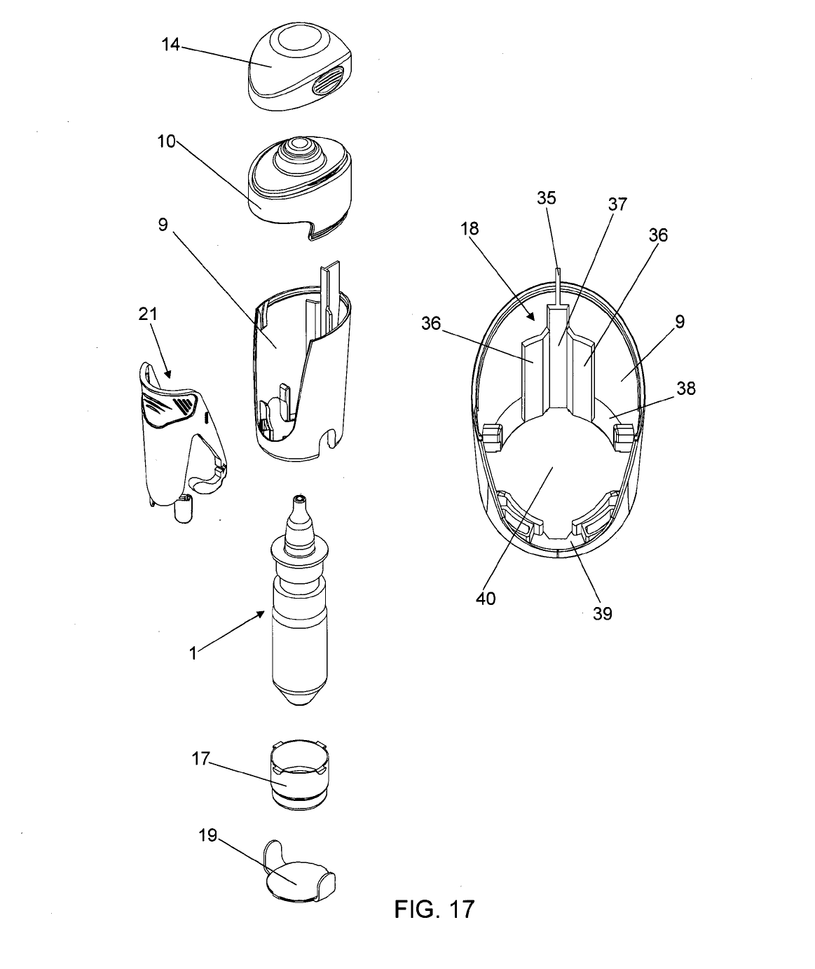

As illustrated in FIGS. 4 to 6, the present mechanism comprises body (8) defined by two parts, a lower grip part (9) and an upper part defined as head (10), coupled together by means (11) existing on its edges which faces each other, these two parts (9-10) equally present an ordinarily elliptic cross-section, the first vertically elongated, whereas the second is substantially short, and its upper side is also closed (12) with a stepped fitting perimeter (13) for a lid (14) which also covers a central, raised and hollowed part (15) which, on the outside partially shows the actuator nozzle (7) and, on the inside, as illustrated in FIGS. 7 to 10, presents means suitable (16) for housing the actuator (5), principally its flange (6), beneath which said flask (2) has its lower end fitted inside an elevator cup (17) which, in turn, is housed inside the grip (9), where there are sliders provided inner housing means (18) for the medicine packaging (1) and its elevator cup (17), and further, said grip (9) is opened from underneath, however, in this part there is fitted a closure component (19), and also said grip has a side, or front side, cut out (20) which also protracts into the head (10), configuring a broadly open side for fastening a trigger (21) with lower means (22) for fitting and fastening inside the grip (9), however, so as to constitute a flexible support point so that said trigger can move angularly, inwardly and outwardly, however, outwardly this movement is limited by lateral means (23), and, further, on each side of the trigger (21) symmetrical actuating means (24) extend downwards and backwards, one on each side, both actuating upwardly to move the elevator cup (17) and, with it, the flask (2), which presses its actuator (5) or flange (6) against the means (16), displacing it according to a sufficient extend so that the pump/valve starts dispensing the medicine through the actuator nozzle (7) in the form of jets, either in spray or any other form.

FIG. 11 shows details of the coupling between the lower part of the grip (9) and the head (10), where it is noted that a reduction in wall and rib thickness configure a male and female profile (25) between the two parts, conferring means for them to be simply fitted together with a certain pressure.

As already stated, the head (10) presents an internal arrangement with suitable means (16) for housing the actuator (5), principally its flange (6) and, accordingly, on the inside there is an ordinarily elliptic wall (26), defined by two opposite sections semicircular in shape (27) which, besides surrounding the base of the raised part (15), also in line with two other opposite sections, but slightly sharp (28), these having internal radial walls (29), one on each side, with the respective notches at a straight angle (30) on the back of the flange (6), whereas on the semicircular parts (27) there are parallel and aligned ribs (31), whose lower ends are aligned with the notches (30), so that they can complete the circular back plane for said flange (6) and, further, below said ribs there is a semicircular cord (32), forming between the latter and the ends of the ribs (31) an invitation gutter (33) for fitting and retaining the flange (6), keeping it sufficiently stable so that flask (2) can be pressed upwards by the trigger (21) displacing it with a sufficient path so that the pump/valve starts dispensing the medicine through the actuator nozzle (7) in the form of jets, either in spray or any other form.

As illustrated in FIG. 16, the elevator cup (17) is cylindrical in shape with a flat bottom and upper edge having preferably four radial, bonnet hip projections (34), all with the flat part upwards, whereas the curved part pointing downwards constitutes a point of support for the actuating means (24) of the trigger (21).

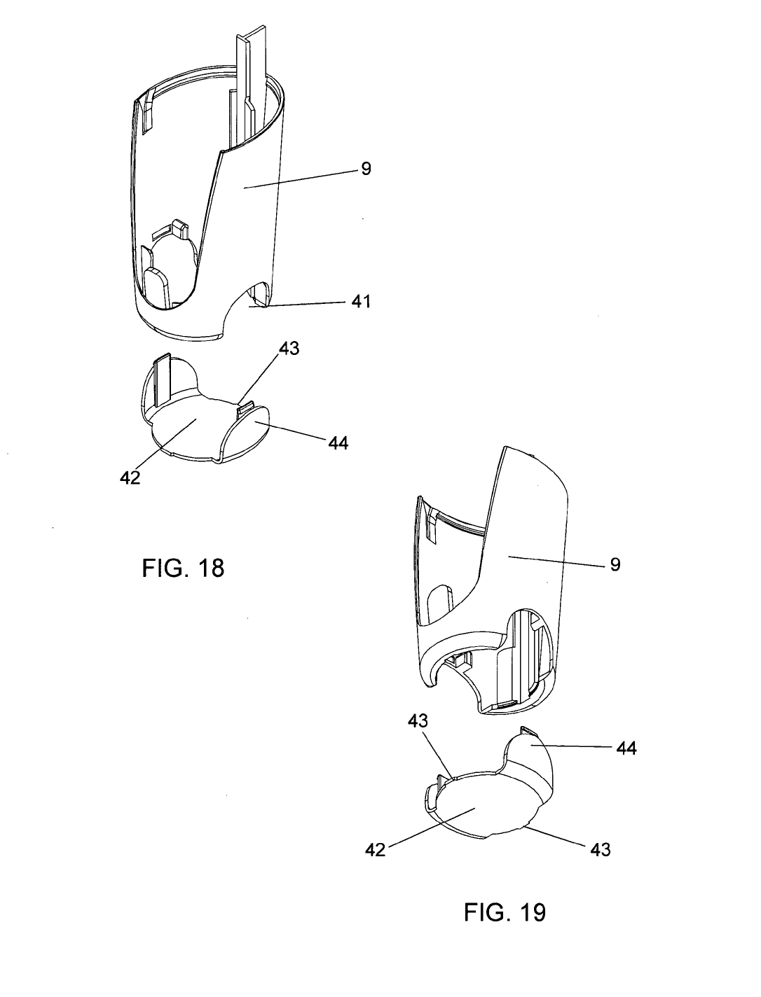

FIG. 17 shows details of the inner sliding housing means (18) for the medicine packaging (1), which are integrated with the inner rear face of the grip (9), where can be noted to exist, initially, a “T” profile (35), which exceeds the height of the grip (9) terminating inside the head (10), whereas on the opposite side, just below the upper edge of the grip (9), said “T” profile expands through two tabs, one on each side (36), between which forming a vertical gutter (37), scaled to receive one of the radial projections (34) of the elevator cup (17), and this winged profile goes as far as the lower edge of the grip (9) which, in turn, projects inwards forming a semicircular tab (38), which also occurs on the opposite side (39), and between these two tabs is a circular opening (40) and also a pair of side notches (41) for fitting the closure (19) which, in turn, is illustrated in FIGS. 18 and 19, where it can be verified that it has a circular part (42) with teeth (43) and latches turned upwards with inner locks (44) which fit into the corresponding parts of the grip (9), closing that opening (40).

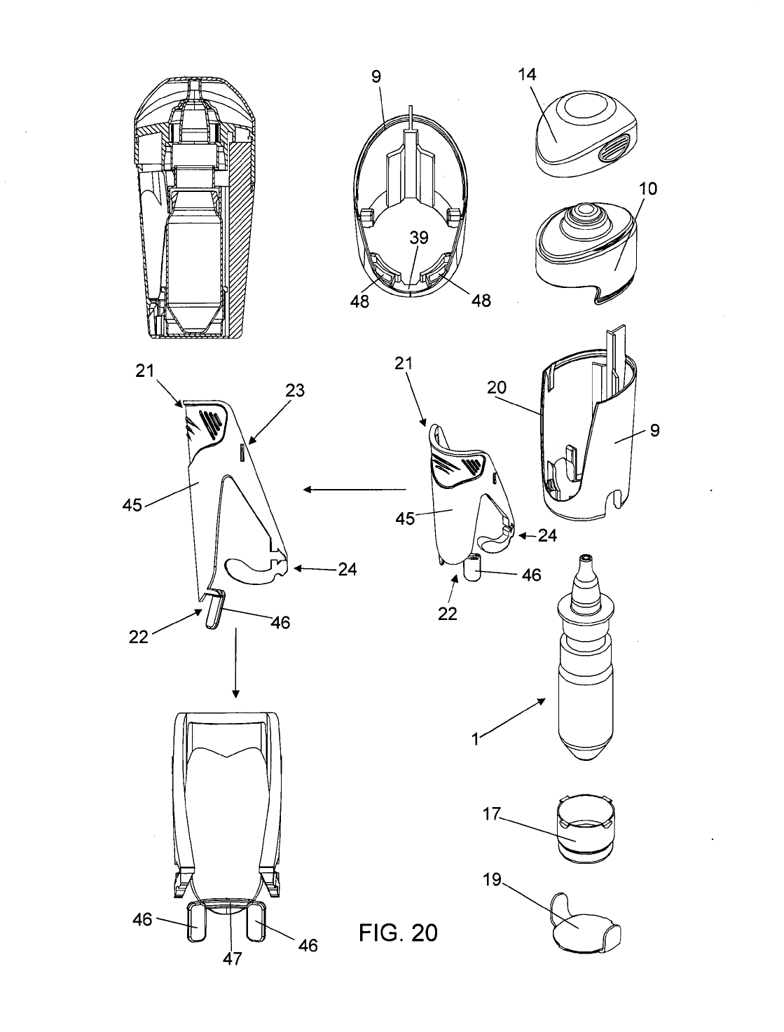

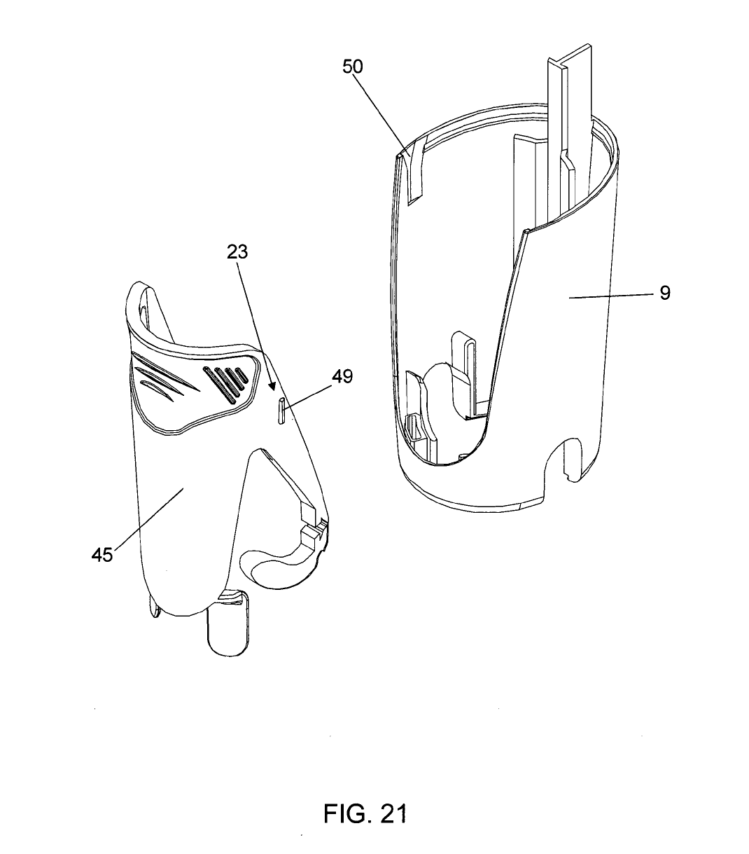

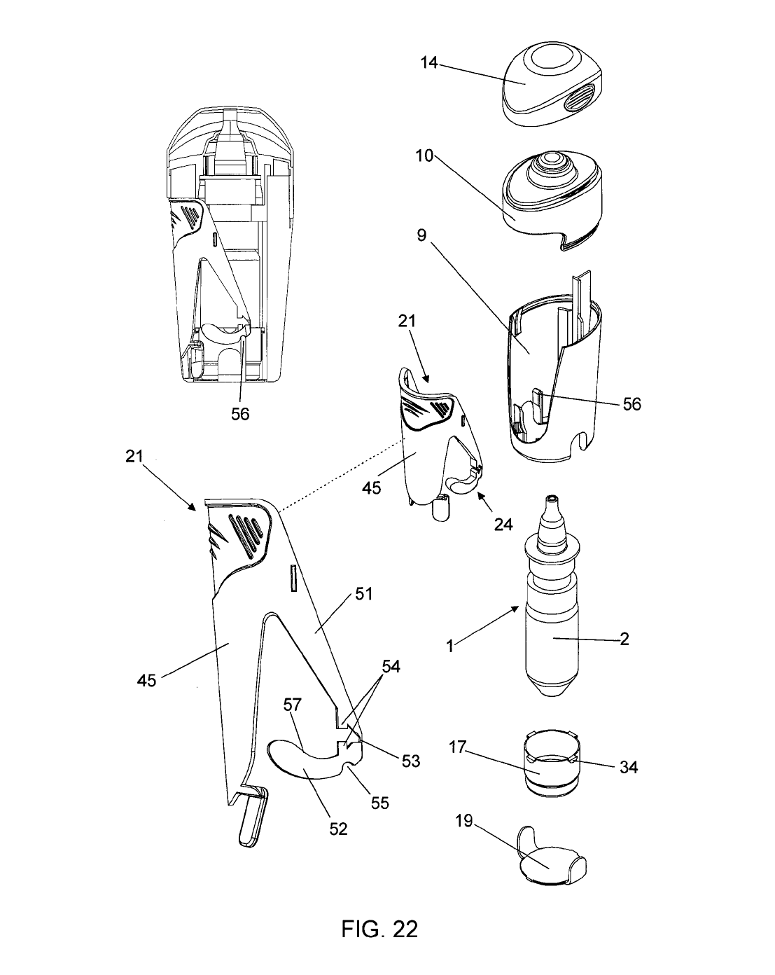

The trigger (21), seen in detail in FIG. 20, is formed by a finger support part (45), anatomically arched in line with the elliptic shape of the grip (9) and closing the notch (20), where its support means (22) are defined by two latches turned downwards (46), both equally interconnected on the rear part of the finger support (45) by a breaker crossbar (47), and said interconnection is made such that said latches are slanted, that is, outwardly, at an angle of less than 180° in relation to the finger support (45), however, this difference disappears when said latches (46) are fitted into the corresponding receptacle (48) existing on the tab (39) inside the grip (9), since, accordingly, said latches are slightly forced to align with the grip (9), which tensions the bar (47), keeping the finger support part (9) normally pressed outwards, however, without causing it to release, since the lateral retention means (23), seen in detail in FIG. 21, prevent this from happening and, accordingly, on each side of the finger support (45) there is a tooth (49) cooperating to fit into the cavity (50) existing on the corresponding inner side of the grip (9), where said cavity has an upright side and a slanted side, allowing the tooth (49) to leave when the finger support (45) is displaced inwards, however, oppositely, said tooth is retained inside the cavity (50).

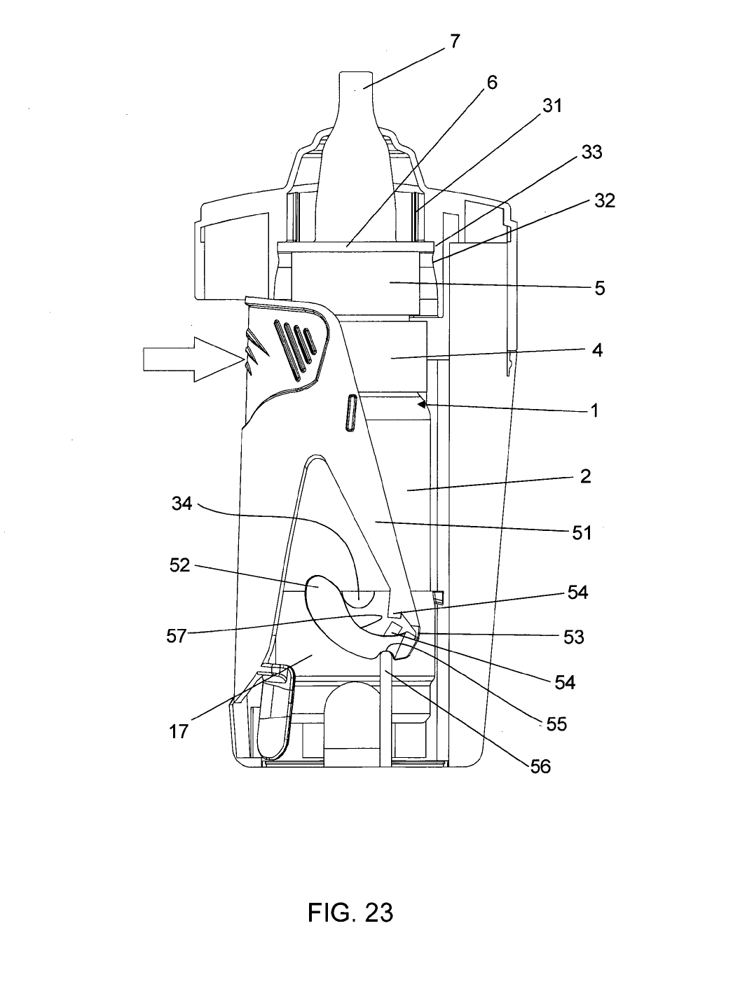

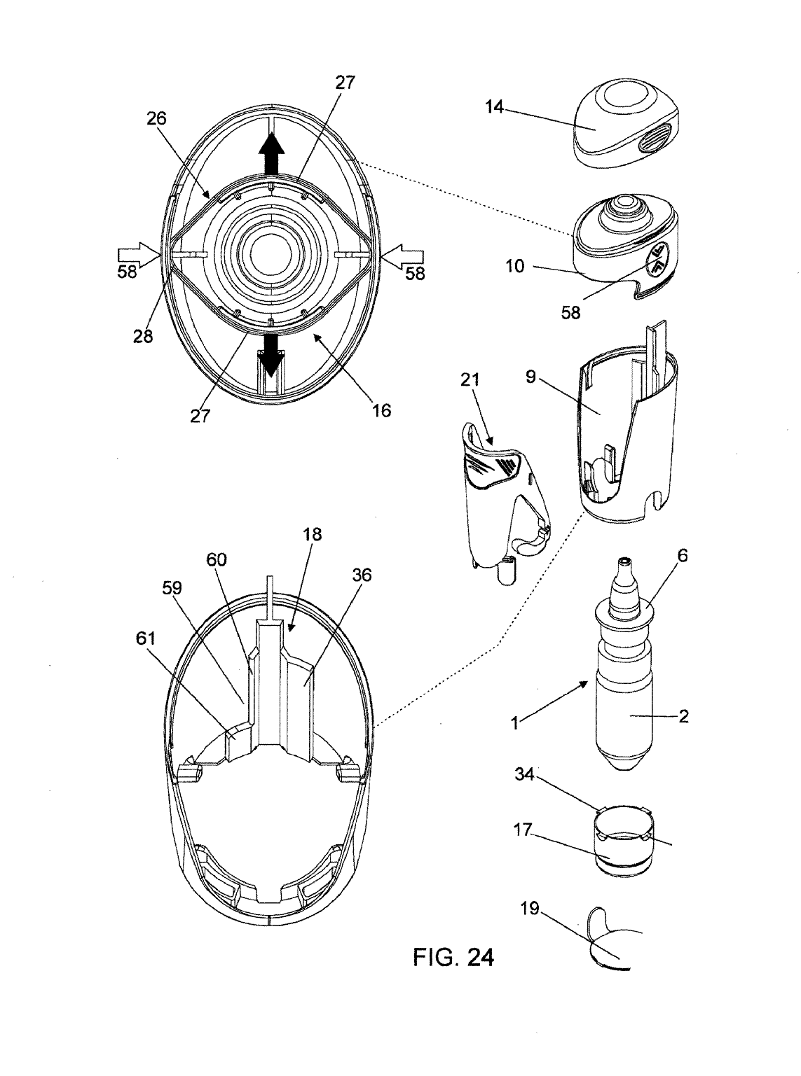

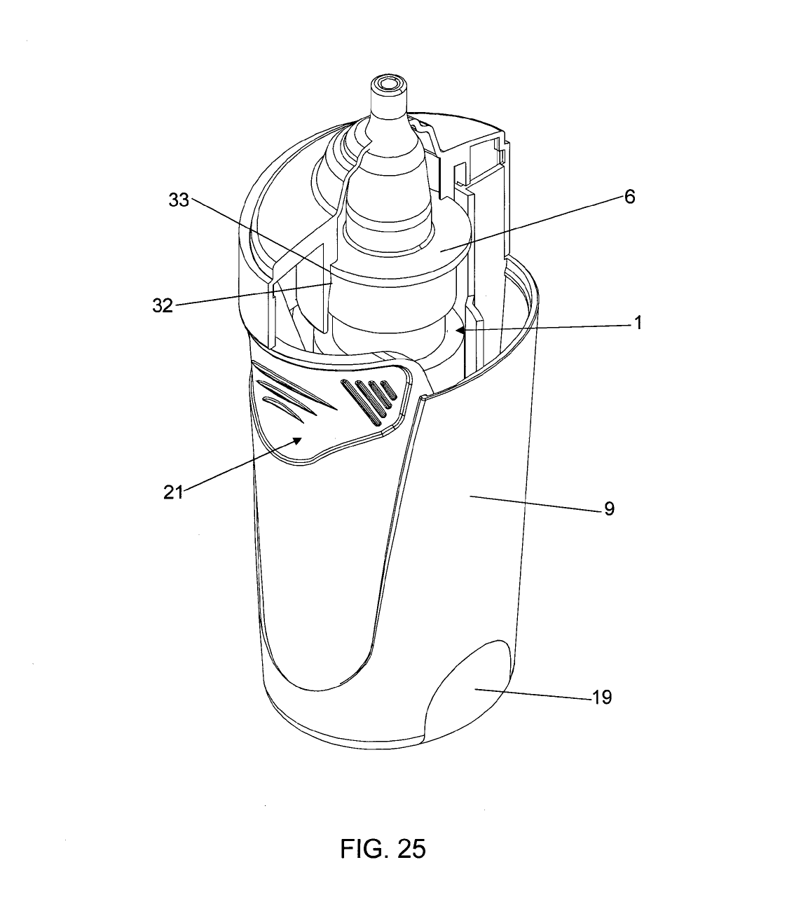

These actuating means (24) seen in detail in FIG. 22, are defined by symmetrical protractions of the finger support (45), such that this set can be positioned embracing the flask (2) and, accordingly, said actuating means (24) comprise slanted arms (51), one on each side, where each lower end has a protraction ordinarily in the form of a horizontal lever (52), where one of its ends is articulately integrate (53) with the corresponding arm (51) and, on the inside portion of said articulation (53), there are projections facing each other (54) limiting the closure angle between the arms (51) and the levers (52) which, further, have lower sides with cavities aligned (55), forming articulated support points for bearing projections (56) existing on the corresponding inner side of the grip (9), where said protraction moves like a seesaw (lever) and, further, its upper face describes a cam surface (57) on which there are supported the corresponding radial projections (34) existing on the edges of said elevator cup (17), such that the latter can be displaced upwards or downwards, as illustrated in FIG. 23, where it can be seen that upon pressing the trigger (21) the lower ends of its arms (51) act upon the corresponding ends of the seesaw protractions (52) and, with this, its cam surface (57) causes said cup (17) to be displaced upwards, supported by the projections (34). This movement has a sufficient extend to drive the actuator (5) of the medicine flask pump (2) with a certain force, dispensing the medicine through the actuator nozzle (7) in the form of jets, either in spray or any other form.

Logically when the trigger (21) ceases to be pressed, it returns to its original position driven by the very spring existing in the metered dose pump (5) of the flask (2). This return goes until the stoppage point defined by the teeth (49) and its cavities (50).

The angle closure limiting projections (54) existing between the arms (51) of the actuating means (21,24) are designed to create a resistance in the lower lever (52) to prevent them from closing beyond a certain extend, as well as for them to have favorable behavior to allow the passage of the projections (34) of the elevator cup (17) containing the medicine packaging (1), since the primary packaging set is introduced in the outer-boby upwardly, and must pass between the levers (52) for said projections (34) to be positioned on the cam surface (57). Therefore, this allows the present dispensing mechanism, which is an outer-packaging device (duly assembled), to receive in its inside portion a set defined as a medicine packaging (1) (duly filled and assembled with the metered dose pump) without the need for adjustments or further assembly.

The movement path of the elevator cup (17) may be influenced to increase or decrease its extent and also to increase or decrease its strength on the level point of the part (52) keeping the same arrangement of the trigger drive assembly (21), only by changing the position of the bearing point (55) along the length of the protraction (52).

Optionally, as illustrated in FIGS. 24 and 25, the assembly is manufactured to receive a flask refill (2) and, accordingly, certain modifications are made on the grip (9) and mainly on the head (10), where the elliptic wall (26) is flexible enough to allow that towards its greater axis—open arrows—its sharpest blunt ends (28) can come together when the head (10) is pressed in the regions that constitutes opposite finger supports (58)—open arrows—causing the semicircular sections to move apart (27)—solid arrows—and, consequently, the flange (6) is released from the gutter (33) formed above the rib (32), allowing said flask to drain downwards along the grip (9) which, has one of its wings (36) vertically cut out (59), so as to have two remaining sections, one vertical (60) and the other lower (61), sufficient for the cup (17) to be displaced and to have its projections (34) free, so that the assembly can leave from underneath, where the closure (19) has been previously removed.

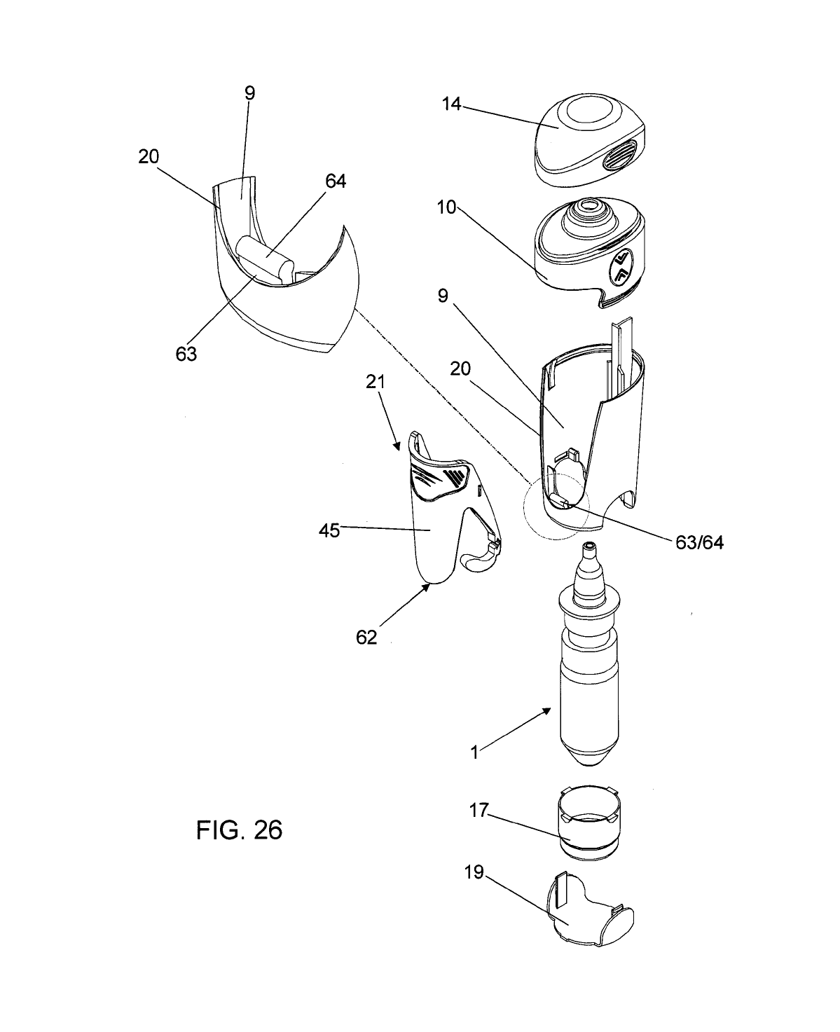

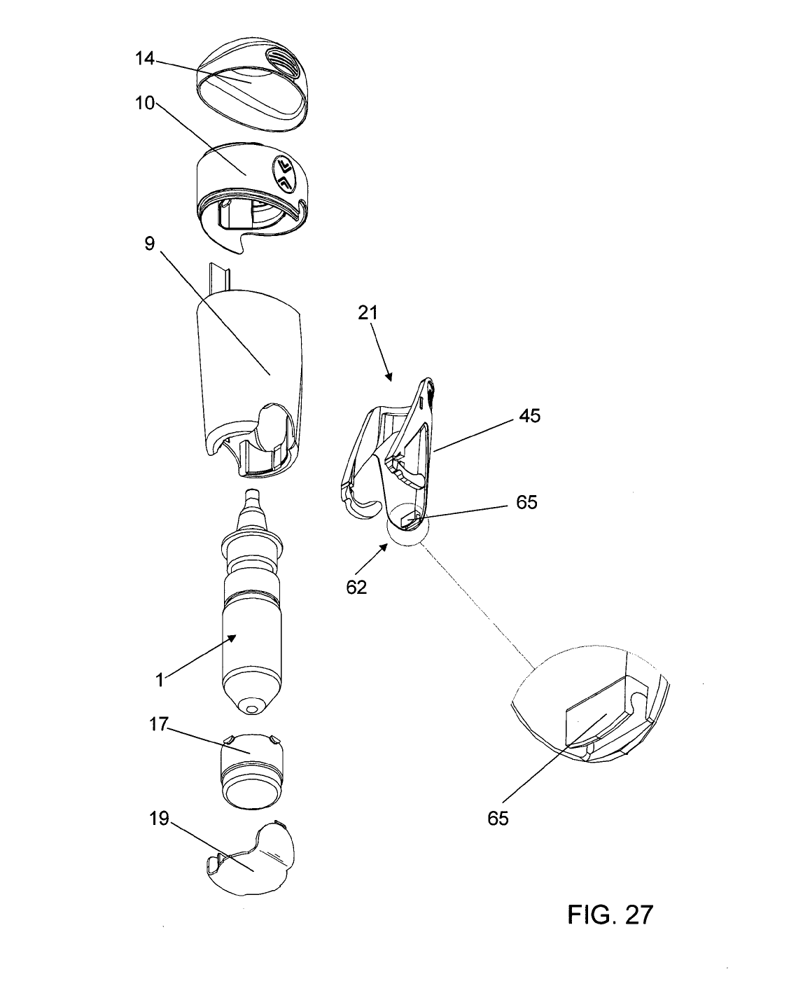

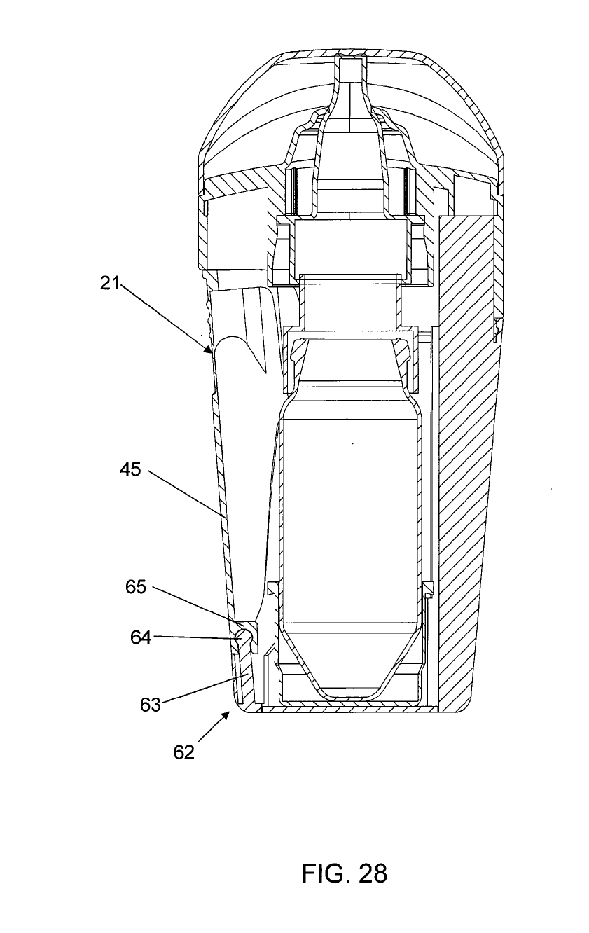

As illustrated in FIGS. 26, 27 and 28, in another preferred embodiment, the trigger includes modified lower means of articulated support (62) defined, initially, by a vertical projection (63) that originates on the inner side of the grip (9) rise slightly above the lowest edge of the opening (20) of the grip, where said projection has a cylindrical upper edge (64), forming a point of articulated support for another female projection (65) existing inside the finger support (45) of the trigger (21), this projection having a suitable fitting to penetrate with a certain pressure and in an articulated manner into the cylindrical part (63).

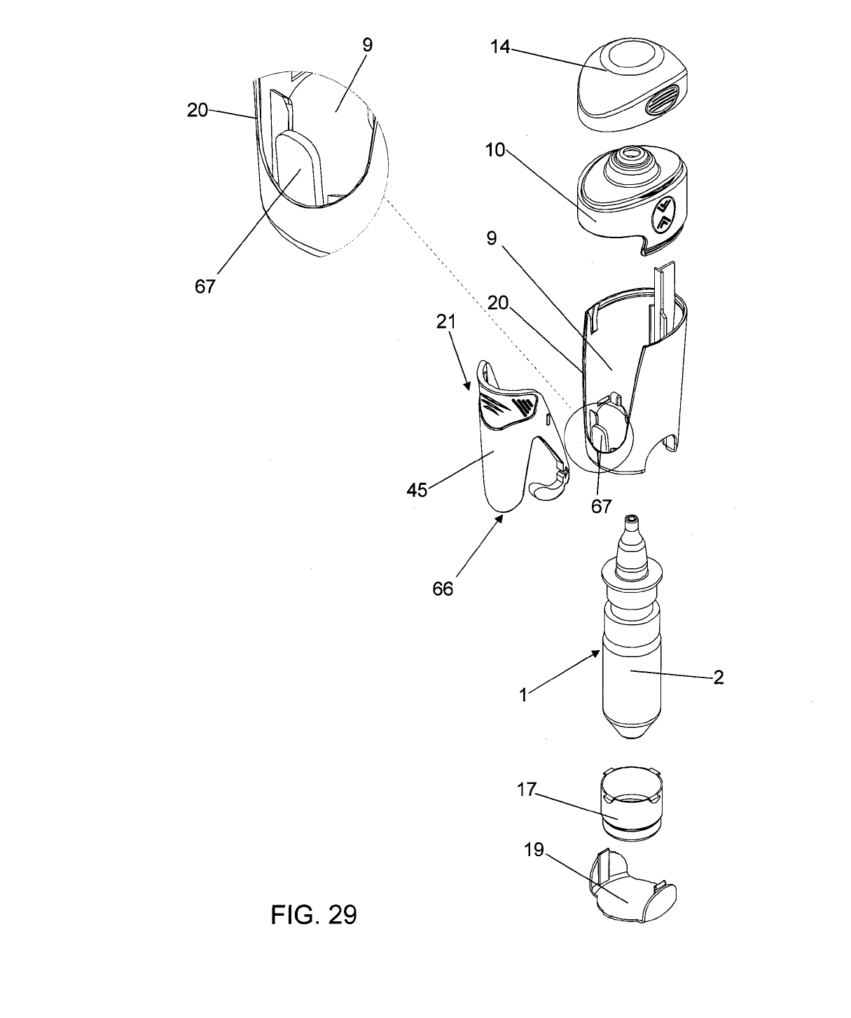

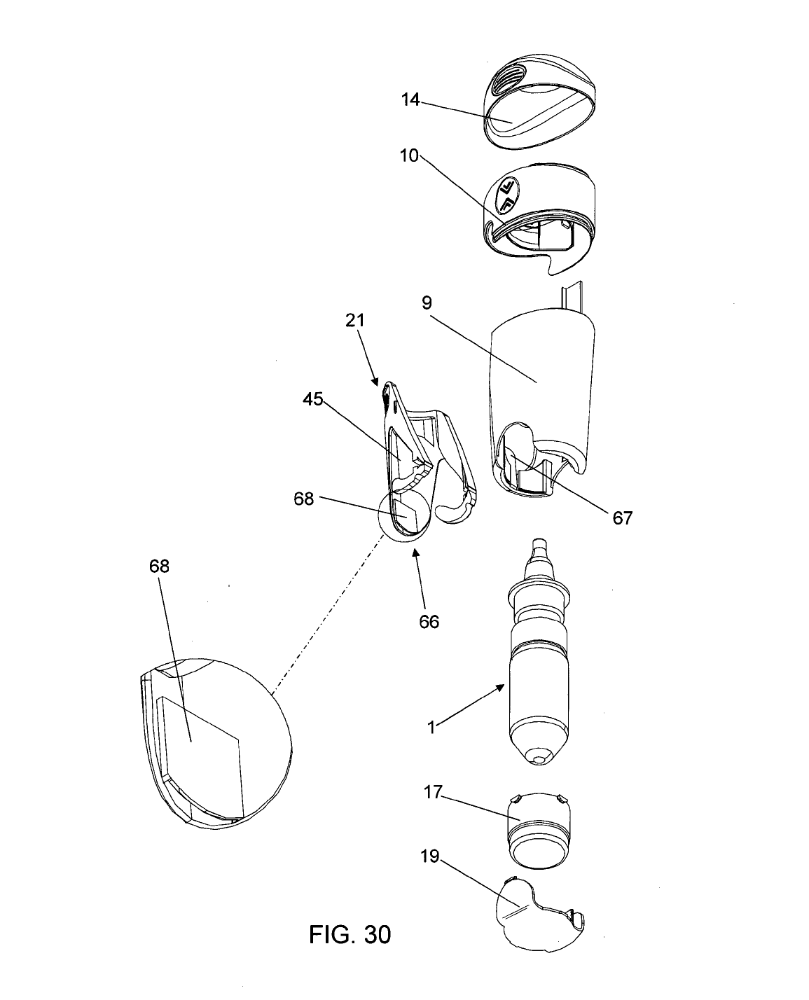

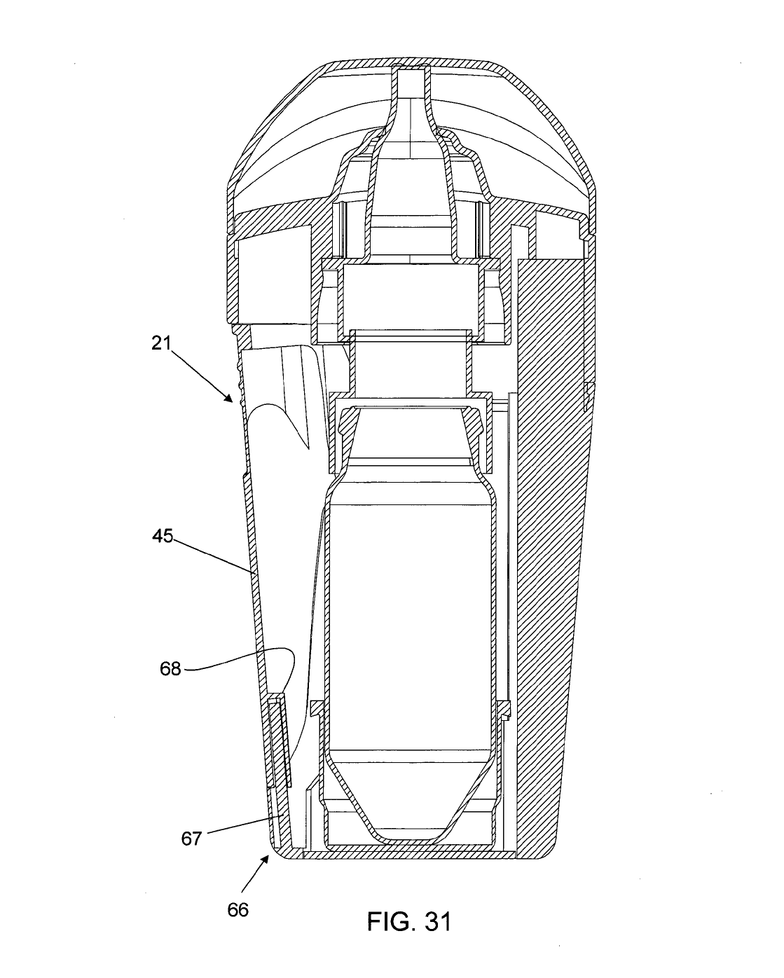

In another preferred embodiment, illustrated in detail in FIGS. 29, 30 and 31, the trigger includes modified lower means of articulated support (66) defined, initially, by a vertical latch (67), substantially flexible, that originates on the inner side of the grip (9) rise slightly above the lowest edge of the opening (20), where said latch is guided to penetrate into a receptacle (68) existing on the inner side of the lower end of the finger support (45) of the trigger (21), such that the latter can remain stable, however, in an articulated manner, since said latch (66) is sufficiently flexible so that the trigger can sustain those movements already referred to during the working of the assembly.

1) MEDICINE DISPENSING DEVICE, of the sort for use with a traditional medicine packaging (1), comprised by a flask (2), whose base or bottom (3) has a profile with any geometry, being it ogival, rounded or straight, whereas the upper part of said flask has a respective lid (4) which functions as an assembly base, having on its upper part an integrated actuator (5) with a median flange (6) and a dispensing portion(6), inside said actuator (5) being assembled a set what configures a dosing pump/valve (not illustrated) mechanism for dispensing the medicine contained in the packaging (1); characterized by comprising a body (8) defined by two parts, a lower grip part (9) and an upper part defined as head (10), coupled together by means (11) existing on its edges which faces each other, these two parts (9-10) equally present an ordinarily elliptic cross-section, the first vertically elongated, whereas the second is substantially short, and its upper side is also closed (12) with a stepped fitting perimeter (13) for a lid (14) which also covers a central, raised and hollowed part (15) which, on the outside partially shows the actuator nozzle (7) and, on the inside, as illustrated in FIGS. 7 to 10, presents means suitable (16) for housing the actuator (5), principally its flange (6), beneath which said flask (2) has its lower end fitted inside an elevator cup (17) which, in turn, is housed inside the grip (9), where there are sliders provided inner housing means (18) for the medicine packaging (1) and its elevator cup (17), and further, said grip (9) is opened from underneath, however, in this part there is fitted a closure component (19), and also said grip has a side, or front side, cut out (20) which also protracts into the head (10), configuring a broadly open side for fastening a trigger (21) with lower means (22) for fitting and fastening inside the grip (9), however, so as to constitute a flexible support point so that said trigger can move angularly, inwardly and outwardly, however, outwardly this movement is limited by lateral means (23), and, further, on each side of the trigger (21) symmetrical actuating means (24) extend downwards and backwards, one on each side, both actuating upwardly to move the elevator cup (17) and, with it, the flask (2), which presses its actuator (5) or flange (6) against the means (16), displacing it according to a sufficient extend so that the pump/valve starts dispensing the medicine through the actuator nozzle (7) in the form of jets, either in spray or any other form.

2) MEDICINE DISPENSING DEVICE, according to claim 1, characterized wherein the coupling between the lower grip part (9) and the head (10) is realized by reducing the wall and rib thickness that configures a male and female profile (25) between the two parts, conferring means for them to be simply fitted together with a certain pressure.

3) MEDICINE DISPENSING DEVICE, according to claim 1, characterized wherein the means (16) for housing the actuator (5), principally its flange (6) and, accordingly, on the inside there is an ordinarily elliptic wall (26), defined by two opposite sections semicircular in shape (27) which, besides surrounding the base of the raised part (15), also in line with two other opposite sections, but slightly sharp (28), these having internal radial walls (29), one on each side, with the respective notches at a straight angle (30) on the back of the flange (6), whereas on the semicircular parts (27) there are parallel and aligned ribs (31), whose lower ends are aligned with the notches (30), so that they can complete the circular back plane for said flange (6) and, further, below said ribs there is a semicircular cord (32), forming between the latter and the ends of the ribs (31) an invitation gutter (33) for fitting and retaining the flange (6).

4) MEDICINE DISPENSING DEVICE, according to claim 1, characterized by an elevator cup (17) being cylindrical and flat-bottomed, its upper edge preferably having four radial, bonnet hip projections (34), all having the flat part turned upwards, whereas the curved part turned downwards constitutes a point of support for the actuating means (24) of the trigger (21).

5) MEDICINE DISPENSING DEVICE, according to claim 1, characterized by inner sliding housing means (18) for the medicine packaging (1), which are integrated with the inner rear face of the grip (9), where can be noted to exist, initially, a “T” profile (35), which exceeds the height of the grip (9) terminating inside the head (10), whereas on the opposite side, just below the upper edge of the grip (9), said “T” profile expands through two tabs, one on each side (36), between which forming a vertical gutter (37), scaled to receive one of the radial projections (34) of the elevator cup (17), and this winged profile goes as far as the lower edge of the grip (9) which, in turn, projects inwards forming a semicircular tab (38), which also occurs on the opposite side (39), and between these two tabs is a circular opening (40) and also a pair of side notches (41) for fitting the closure (19).

6) MEDICINE DISPENSING DEVICE, according to claim 1, characterized wherein the closure (19) comprises a circular part (42) with teeth (43) and latches turned upwards with inner locks (44) which fit into the corresponding parts of the grip (9), closing that opening (40) and the side notches (41).

7) MEDICINE DISPENSING DEVICE, according to claim 1, characterized wherein the trigger is formed by a finger support part (45), anatomically arched in line with the elliptic shape of the grip (9) and closing the notch (20), where its support means (22) are defined by two latches turned downwards (46), both equally interconnected in the rear part of the finger support (45) by a breaker crossbar (47), which interconnection is made such that said latches are slanted, that is, on the outside, form an angle less than 180° with the finger support (45), however, this difference disappears when said latches (46) are fitted into the corresponding receptacle (48) existing on the tab (39) inside the grip (9), since, accordingly, said latches are slightly forced to align with the grip (9), which tensions the bar (47), keeping the finger support part (9) normally pressed outwards, however, with movement limited by the side retention means (23).

8) MEDICINE DISPENSING DEVICE, according to claim 1, characterized wherein said lateral retention means (23) are formed on each side of the finger support (45), where there is a tooth (49) coopering to fit into the cavity (50) existing on the corresponding inside of the grip (9), and said cavity (50) has an upright side and a slanted side, where the latter allows the tooth (49) to leave when the finger support (45) is displaced inwards, however, oppositely, said tooth is retained inside the cavity (50) by the straight side.

9) MEDICINE DISPENSING DEVICE, according to claim 1, characterized wherein the actuating means (24) comprise symmetrical protractions of the finger support (45), such that this set can be positioned embracing the flask (2) and, accordingly, said actuating means (24) comprise slanted arms (51), one on each side, where each lower end has a protraction ordinarily in the form of a horizontal lever (52), where one of its ends is articulately integrated (53) with the corresponding arm (51) and, on the inside portion of said articulation (53), there are projections which face each other (54) limiting the closure angle between the arms (51) and the levers (52) which, further, have lower sides with cavities aligned (55) forming articulated support points for the bearing projections (56) existing on the corresponding inner side of the grip (9), where said protraction moves like a seesaw (lever) and, further, its upper face describes a cam surface (57) on which there is supported the corresponding radial projections (34) existing on the edges of said elevator cup (17).

10) MEDICINE DISPENSING DEVICE, according to claim 1, characterized wherein the elevator cup (17) is displaced upwards at the moment in which the trigger (21) is pressed inwards and the lower ends of its arms (51) act upon the corresponding ends of the seesaw protractions (52) and, with this, its cam surface (57) makes said cup (17) be displaced upwards supported by the projections (34).

11) MEDICINE DISPENSING DEVICE, according to claim 10, characterized wherein the upward displacement of the elevator cup (17) has a sufficient extend to drive the actuator (5) of the medicine flask pump (2), dispensing the medicine through the actuator nozzle (7) in the form of jets, either in spray or any other form.

12) MEDICINE DISPENSING DEVICE, according to claim 10, characterized wherein the trigger (21) returns to its original position driven by the very spring existing in the metered dose pump (5) of the flask (2) and this return goes until the stoppage point defined by the teeth (49) and its cavities (50).

13) MEDICINE DISPENSING DEVICE, according to claim 9, characterized wherein the movement path of the elevator cup (17) is influenced to increase or decrease its extent and also to increase or decrease its strength on the level point of the part (52) maintaining the same arrangement of the drive assembly of the trigger (21), the only by changing the position of the bearing point (55) along the length of the protraction (52).

14) MEDICINE DISPENSING DEVICE, according to claim 1, characterized wherein, optionally, the assembly is manufactured to receive a refill of the flask (2) and, accordingly, certain changes are made to the grip (9) and mainly on the head (10) where the elliptic wall (26) is flexible enough to allow that towards its greater axis, as its sharpest ends (28) can come together when the head (10) is pressed in regions that constitute opposite finger supports (58) causing the semicircular sections to move apart (27) and, consequently, the flange (6) is released from the gutter (33) formed above the rib (32), allowing said flask to drain downwards along the grip (9) which, in turn, has one of the wings (36) cut out vertically (59), so as to have two remaining sections, one vertical (60) and the other lower (61), sufficient for the cup (17) to be displaced and to have its projections (34) free so that the assembly can leave from underneath, where the closure (19) has been previously removed.

15) MEDICINE DISPENSING DEVICE, according to claim 1, characterized wherein, in another preferred embodiment, the trigger includes modified lower means of articulated support (62) defined, initially, by a vertical projection (63) that originates on the inner side of the grip (9) rise slightly above the lowest edge of the closure (20) of the grip, where said projection has a cylindrical upper edge (64), forming a point of articulated support for another female projection (65) existing on the inside of the finger support (45) of the trigger (21), said projection having a suitable fitting for penetrating with a certain pressure and in an articulated manner into the cylindrical part (63).

16) MEDICINE DISPENSING DEVICE, according to claim 1, characterized wherein, in another preferred embodiment, the trigger includes modified lower means of articulated support (66) defined, initially, by a vertical latch (67), substantially flexible, that originates on the inner side of the grip (9) rise slightly above the lowest edge of the opening (20), where said latch is guided to penetrate into a receptacle (68) existing on the inner side of the lower end of the finger support (45) of the trigger (21).
